(19) United States
(12) Patent Application Publication
(10) Pub. No.: US 2015/0039088 A1
(43) Pub. Date: Feb. 5, 2015

(54) MIDDLE EAR PROSTHESIS

(71) Applicant: MOR RESEARCH APPLICATIONS LTD., Tel Aviv (IL)

(72) Inventor: Salim Mazzawi, Nazareth (IL)

(73) Assignee: MOR RESEARCH APPLICATIONS LTD, Tel Aviv (IL)

(21) Appl. No.: 14/385,956

(22) PCT Filed: Mar. 14, 2013
(86) PCT No.: PCT/IL2013/050236

§ 371 Date: Sep. 17, 2014

(30) Foreign Application Priority Data
Mar. 19, 2012 (IL) 218715

(51) Int. Cl. A61F 2/18

(52) U.S. Cl. CPC A61F 2/18; A61F 2002/183
USPC 623/10

(57) ABSTRACT

A middle ear prosthesis (40) is provided for mounting in the middle ear (4). The middle ear prosthesis comprises a shaft (42) having a distal end configured for the introduction into the inner ear (8) through an aperture (30) formed in a footplate (23) of a stapes bone and in a membrane of an oval window of the inner ear, by moving the shaft in an introduction direction, the shaft being configured for transmitting sound wave vibrations to the inner ear. The prosthesis further comprises a coupling portion (46), for coupling, the shaft to at least one of the ossicles (17) so as to allow transmitting thereby sound wave vibrations from the ossicles to the shaft; and a stop member (48) spaced from the distal end of the shaft and configured for bearing against the remaining portions thereby limiting the movement of the shaft in the introduction direction.

### FIELD OF THE DISCLOSED SUBJECT MATTER

This presently disclosed subject matter relates to a middle ear prosthesis. More particularly, the presently disclosed subject matter relates to a stapes prosthesis.

### BACKGROUND DESCRIPTION

The anatomy and physiology of the ear is typically referred to as three parts: the outer ear, the middle ear and the inner ear, all of which are responsible for transmitting sound waves which are converted to neural activity to be sent to the brain through the auditory portion of the eighth cranial nerve.

FIGS. 1A and 1B, show a cross section of the outer ear 2, the middle ear 4 and the inner ear 6. The middle ear 4 is the portion of the ear defined between the tympanic membrane 12 (typically referred to as the ‘eardrum’) and the oval window 14 of the cochlea 8 which is located in the inner ear 6. The cavity of the middle ear contains the three ossicles (bones) the malleus 15 (hammer), incus 17 (anvil), and stapes 19 (stirrup), which are coupled to one another forming together an ossicular chain. The ossicular chain transfers and amplifies sound wave vibrations of the eardrum 12 to the fluid and membranes of the cochlea in the inner ear 6. In particular the stapes 19 includes an arch portion 21 which is coupled to the incus 17, and a footplate portion 23 which is coupled to the membrane 13 covering the oval window 14 for vibrating thereof.

Hearing losses are sometimes caused by ossicular dysfunction, for example, due to a disease such as Otosclerosis, congenital malformation or trauma. There are known surgical procedures for resorting hearing losses caused by malfunction of the ossicles. Stapedomoty is a surgical procedure in which a stiffed stapes is replaced by prosthesis. During the stapedotomy procedure the arch portion 21 of the stapes bone 19 is removed, a small aperture is drilled in the footplate portion 23 thereof, and through the membrane of the oval window 14.

Referring now to FIG. 2, the stapes prosthesis 25, is typically a piston-like prosthesis having a shaft 26 affixed to coupling means 28, such as a wire crimp. Referring now to FIG. 3, the prosthesis is placed in an aperture 30 formed in the footplate portion 23 and the membrane 13 of the oval window 14, and the coupling means 28 are coupled to the remaining ossicles, typically the incus 17, and sometimes the malleus 15, in such a manner which allows sound vibrations to propagate therethrough, from the eardrum 12 to the inner ear 6, thereby restoring hearing.

There are known stapes or other middle ear prosthesis in the art, for example, US Patent 2007/0055372 discloses middle ear prosthesis comprising a member adapted to supplement an ossicular bone when implanted in an ear. An anatomically conforming structure is secured to the member comprising an upper arcuate portion and a lower arcuate portion, each having near and distal ends. A crimp assist portion is connected to the near ends of the upper and lower arcuate portions so that the upper and lower arcuate portions generally define portions of a circle for partially surrounding a bone. The crimp assist portion extends outside of the circle. The crimp assist portion is of a crimpable metal capable of retaining different shapes so that the crimp assist portion can be crimped to close the structure around the bone without crimping within the circle.

U.S. Pat. No. 7,011,683 discloses a middle ear prosthesis comprising a piston adapted to extend through an oval window when implanted in a human ear. A pair of jaws engages an ossicle when implanted in a human ear. A spring is coupled to the jaws for biasing the jaws toward one another to provide clamping pressure. The jaws are in turn connected to the piston.

During the stapedotomy procedure, positioning the shaft inside the aperture formed in the membrane must be carried out in such a way to allow the vibrations of the prosthesis to be properly transferred to the membrane. Thus, it is required that one end of the shaft is inserted through the membrane and is positioned within the inner ear. On the other hand, however, care should be taken when inserting the piston into the inner ear, so as to preclude causing damage to the inner membranes of the cochlea, which are likely to occur when the shaft is inserted too deeply therein. In addition, following the stapedotomy procedure, during the healing process, the shaft can he displaced further into the inner ear, for example due to an incautious movement of the patient.

FIG. 4 illustrates a common scenario occurring throughout the stapedotomy procedure, in which the entire shaft 26 of the prosthesis 25 is unintentionally introduced into the cochlea 8 through the aperture 30 formed in the oval window 14. This scenario can also occur, after the stapedotomy procedure, for example in case the coupling means 28 disengages the incus 17. In this scenarios the shaft 26 cannot properly transfer vibrations from the ossicles, 17 to the membrane 13 of the oval window 14, and fluids from the cochlea 8 may leak thorough the aperture. Furthermore, while attempting to bring the shaft 26 back to the desired position in the aperture 30, the surgeon can cause damage to the membrane of the oval window.

### SUMMARY OF THE PRESENTLY DISCLOSED SUBJECT MATTER

According to an aspect of the presently disclosed subject matter there is provided middle ear prosthesis for mounting in the middle ear, comprising:

a shaft having a distal end configured for the introduction into the inner ear through an aperture formed in a footplate of a stapes bone and in a membrane of an oval window of the inner ear, by moving the shaft in an introduction direction, the shaft being configured for transmitting sound wave vibrations to the inner ear;

a coupling portion, for coupling, the shaft to at least one of the ossicles so as to allow transmitting thereby sound wave vibrations from the ossicles to the shaft; and

a stop member spaced from the distal end of the shaft and configured for bearing against the remaining portions thereby limiting the movement of the shaft in the introduction direction.

The stop member can be a laterally projecting member or a ring disk.

In addition, the stop member can be mounted on the shaft thereby defining the distal end on first side thereof and an upper portion on a second side thereof, the upper portion being configured to protrude inside the cavity of the middle ear.

The stop member can be further configured to indicate to a surgeon conducting the medical procedure the location of the shaft with respect to the aperture.

The stop member can be mounted on the coupling portion or between the coupling portion and the shaft.

The stop member can be adjustably mounted on the shaft and can be provided with fastening means, for securing thereof in the desired location with respect to the length of the shaft.

According to another aspect of the presently disclosed subject matter there is provided an addition for a middle ear prosthesis comprising a shaft configured for the introduction into the inner ear through an aperture formed in a footplate of a remaining portions of a stapes bone and in a membrane of an oval window of the inner ear, by moving the shaft in an introduction direction, the shaft being configured for transmitting sound wave vibrations to the inner ear; and a coupling portion for coupling the shaft to at least one of the ossicles so as to allow transmitting thereby sound wave vibrations from the ossicles to the shaft, the addition comprising:

a stop member mountable on the prosthesis thereby defining distal end of the shaft configured for the introduction into the inner ear, the stop member being configured for bearing against the remaining portions thereby limiting the movement of the shaft in the introduction direction.

The stop member can be a laterally projecting member or a ring disk such as a rubber stretch ring.

In addition, the stop member can be configured to be mounted on the shaft thereby defining the distal end on first side thereof and an upper portion on a second side thereof, the upper portion being configured to protrude inside the cavity of the middle ear.

The stop member can be further configured to indicate to a surgeon conducting the medical procedure the location of the shaft with respect to the aperture.

The stop member can be configured to be mounted on the coupling portion or between the coupling portion and the shaft.

The stop member can be configured to be adjustably mounted on the shaft and can be provided with fastening means, for securing thereof in the desired location with respect to the length of the shaft.

### BRIEF DESCRIPTION OF THE DRAWINGS

In order to understand the presently disclosed subject matter and to see how it may be carried out in practice, embodiments will now be described, by way of non-limiting example only, with reference to the accompanying drawings, in which:

FIG. 1A is a front cross section schematic view of a human body ear;

FIG. 1B is an enlarged view of the middle ear illustrated in FIG. 1A;

FIG. 2 is a front view of a prior art stapes piston prosthesis;

FIG. 3 is a front cross section schematic view of a middle ear having the stapes piston prosthesis of FIG. 2 implanted therein;

FIG. 4 is a side cross section schematic view of an oval window of the inner ear with the stapes piston prosthesis of FIG. 2 intruding deep into the inner ear;

FIG. 5A is a front view of a stapes piston prosthesis according to one example of the presently disclosed subject matter;

FIG. 5B is a front view of a stapes piston prosthesis according to another example of the presently disclosed subject matter; and

FIG. 6 is a side cross section schematic view of an oval window of the inner ear having the stapes prosthesis of FIG. 5A implanted therein.

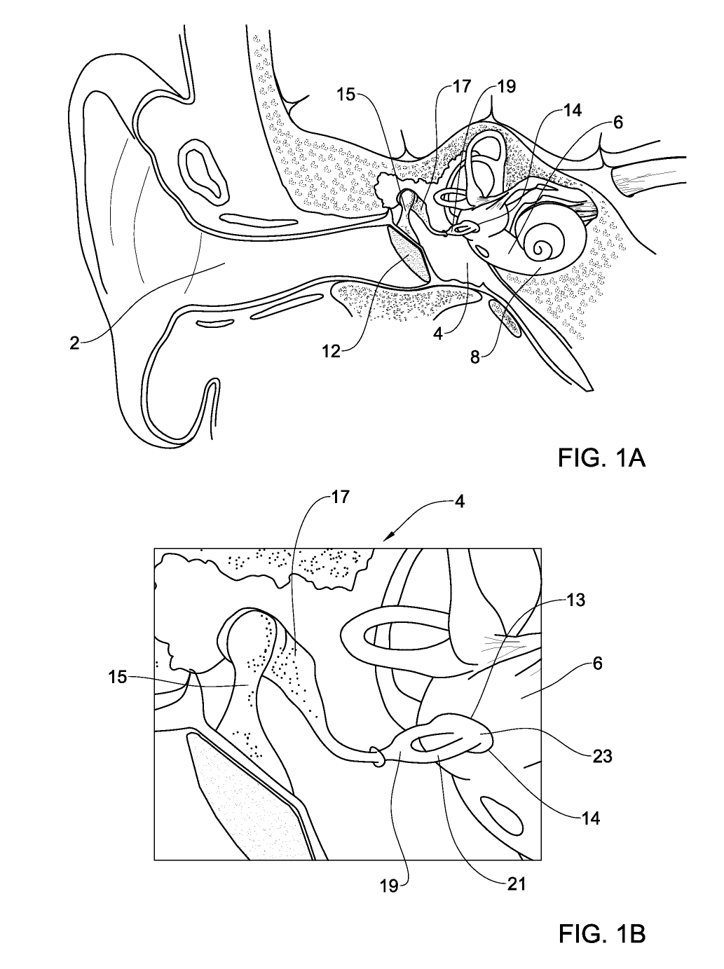

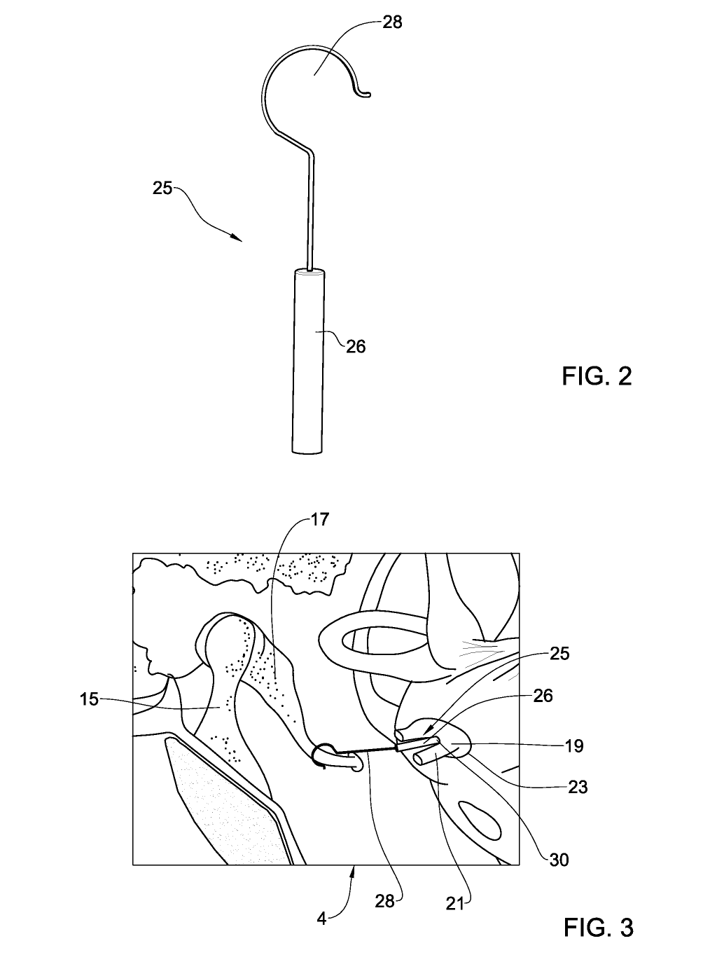

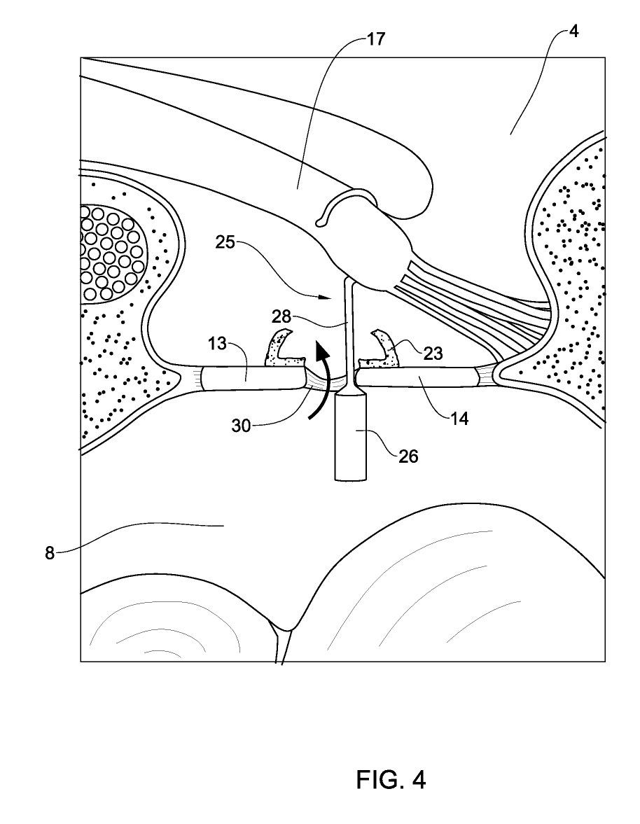

### DETAILED DESCRIPTION OF EXAMPLES

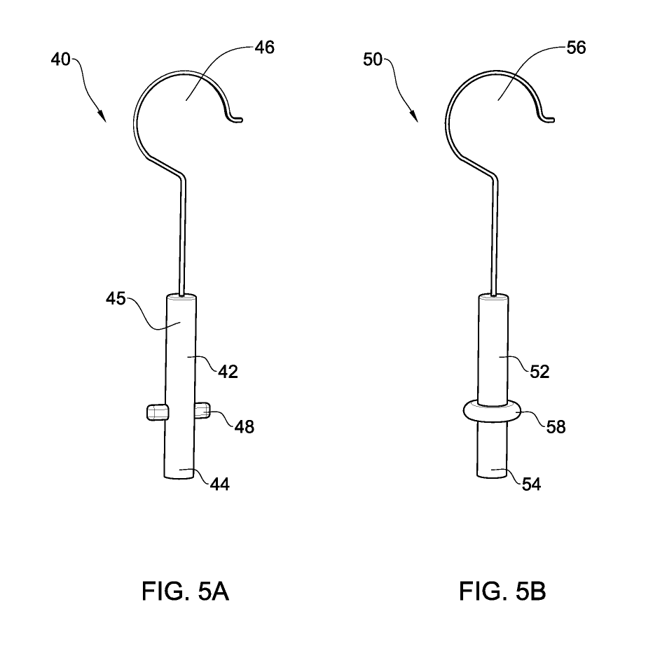

FIG. 5A illustrates a middle ear prosthesis 40 comprising a shaft 42 having a distal end 44, for introducing into an aperture formed in a footplate portion of a stapes and in an oval window membrane. The shaft 42, here illustrated as a cylindrical piston, is configured for transmitting sound wave vibrations to the footplate and the membrane, and thereby to the inner ear. The prosthesis 40 further includes a coupling portion 46 for coupling thereof to one of the ossicles in the middle ear. The coupling portion 46 can be, for example, a hook or a wire crimp configured for coupling to one of the ossicles, for example, the incus, in such a way that sound vibrations therefrom are transferred to the shaft 42, and thereby to the inner ear. The coupling portion can include a strut to ease the handling of the prosthesis.

The prosthesis 40 further includes a stop member 48 spaced from the distal end 44 of the shaft 42. The stop member 48 is configured for bearing against the footplate portion of the stapes thereby the stop member limits the movement of the shaft 42 in the direction of the introduction e.g. the direction towards the inner ear. This way, the stop member 48 precludes a possible deep intrusion of the shaft 42, during the stapedotomy procedure or thereafter, for example in case the coupling portion 46 disengages the incus.

The stop member 48, according to this example, is in a form of a laterally projecting member, configured to engage the membrane of the oval window, at least on one side of the aperture thereby limiting the movement of the prosthesis toward the inner ear.

The stop member 48 is disposed at a predetermined position along the length of the shaft 42, thereby defining the distal end 44 on one side thereof and an upper end 45 on a second side thereof. The distal end 44 is configured to be inserted to the cochlea through the aperture formed in the footplate while the upper end 45 protrudes outside the footplate in the middle ear cavity. It is appreciated that the position of the stop member 48 along the length of the shaft 42, is determined in accordance with the required portion of the piston to be introduced in the membrane so as to allow properly transmitting sound wave vibrations to the inner ear.

FIG. 5B shows a middle ear prosthesis 50 which is substantially the same as the prosthesis 40 of FIG. 5A, and includes a shaft 52, a coupling portion 56 and a stop member 58. The stop member 58 according to this example is a ring disk mounted on the shaft 52, thereby defining a distal end 54 configured to be introduced to the aperture in a footplate and oval window, an upper end protruding outside the footplate and the membrane in the cavity of the middle ear.

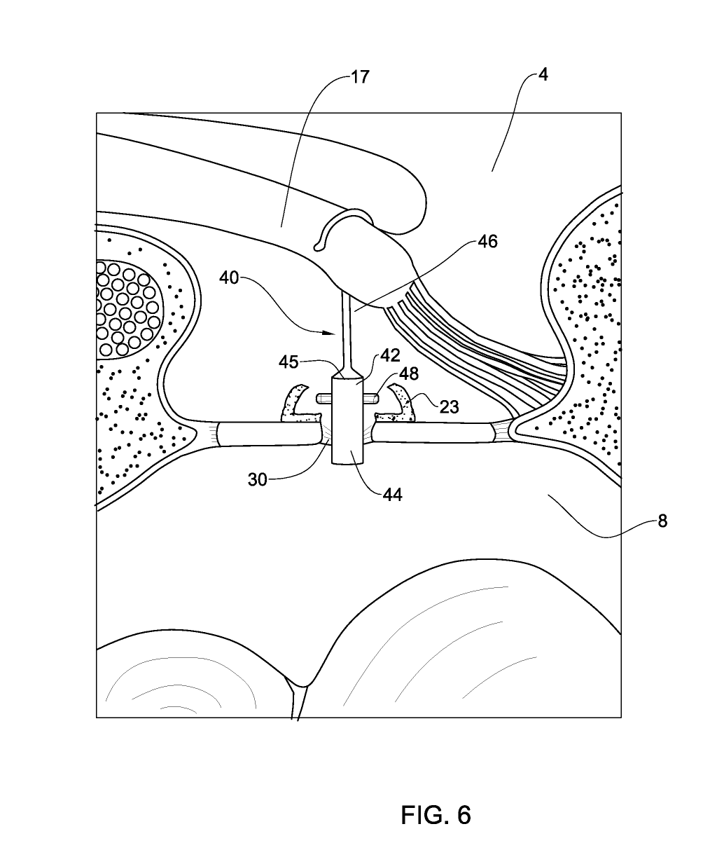

Referring now to FIG. 6, during a procedure, such as a stapedotomy, an aperture 30 is formed in the footplate and the membrane 13 of the oval window 14. A prosthesis, such as the prosthesis of FIG. 5A is introduced in the aperture 30, by inserting the distal end 44 of the shaft 42 therein, in a direction which is typically transverse to the membrane 13, towards the inner portions of the cochlea 8. The prosthesis 40 can be further pushed into the aperture 30, until the stop member 48 bears against the footplate portion and or the membrane 13. At that position, the stop member 48 limits the movement of the shaft 42 in the direction of the introduction, i.e. towards the inner portions of the cochlea, thus precluding damage thereof by the shaft 42.

After the procedure the shaft is free to vibrate inside the aperture 30, thereby transmitting sound wave vibrations into the inner ear. The stop member 48, thus, can abut the footplate during the vibration, or alternatively, can be disposed above the footplate without engaging thereon. However, in any case the stop member 48 limits the movement of the shaft 42 inside the inner ear, thereby precluding damage of the cochlea.

It is appreciated that disposition of the stop member 48 with respect to the length of the shaft 42 can be determined in accordance with the required length of the distal end 44. That is to say, the length of the distal end can be determined in accordance with the width of the footplate and the membrane 13 through which the shaft is introduced, and the required length thereof to be positioned inside the cochlea 8, such that sound vibrations can be effectively transmitted to the inner ear. When the distal end 44 is properly positioned inside the membrane 13 and the cochlea 8 the piston can properly transmit sound waves vibrations to the inner ear without escaping the aperture 30. The stop member 48, on the other hand, limits the piston from further being pushed into the cochlea 8.

It is appreciated that according to one example, the disposition of the stop member 48 can be adjusted with respect to the length of the shaft 42. This way, the size of the distal end 44 to be introduced through the aperture 30, is adjusted as desired, for example, in accordance with the exact middle and inner ear's anatomy of the patient. The stop member accordingly, can be provided with fastening means (not shown) for fastening thereof to the desired location along the shaft 42. According to one example the stop member can be a rubber stretch ring mountable on the piston, with at least a portion thereof configured to engage the footplate, thereby limit the movement of the piston in the direction of the inner ear.

It is further appreciated that according to another example the stop member can be positioned on the coupling portion 46 or between the coupling portion and the shaft 42. In such a case, however, the shaft 42 can be configured to be fully introduced through the aperture 30. Accordingly, the piston portion 30 is coupled to a coupling portion 46 which in return extends from one of the remaining ossicles, to the shaft 42 disposed in the aperture 30.

According to one example, a stop member in a form of a ring disk can be provided, for mounting on a shaft of a conventional middle ear prosthesis. In this Example, the stop member can be an add-on, which can be mounted and affixed to the shaft at a desired location thereon. The stop member, thus, defines a distal end on one end of the shaft, to be introduced into an aperture formed in the footplate and the oval window. The stop member defines an upper end on the other end of the shaft, which protrudes inside the cavity of the middle ear, and is coupled to the ossicles, for example, via a coupling means.

It is noted that according to another aspect of the presently disclosed subject matter, the stop member serve as an indicator, assisting the surgeon in identifying the exact location of the prosthesis with respect to the footplate. That is to say, when looking at the prosthesis from the top side of the middle ear, determining whether the shaft is positioned inside the aperture formed in the footplate, can be a difficult task. However, when a stop member is provided, when the stop member is positioned on top of the footplate, due to the diameter thereof, which is larger than that of the shaft, it is easier to bring the shaft to the desired position so it can be introduced into the aperture. This way, the stop member assists in positioning the prosthesis in the desired position

Those skilled in the art to which the presently disclosed subject matter pertains will readily appreciate that numerous changes, variations, and modifications can be made without departing from the scope of the invention, mutatis mutandis.

1. A middle ear prosthesis for mounting in a middle ear having ossicles including remaining portions of a stapes bone with a footplate, the middle ear being disposed adjacent to an inner ear having an oval window with a membrane, the middle ear prosthesis comprising:
a shaft having a distal end configured for the introduction into the inner ear through an aperture formed in the footplate of said remaining portions and in the membrane, by moving the shaft in an introduction direction, said shaft being configured for transmitting sound wave vibrations to the inner ear;
a coupling portion, for coupling, said shaft to at least one of the ossicles so as to allow transmitting thereby sound wave vibrations from the at least one of the ossicles to said shaft; and
a stop member spaced from said distal end of the shaft and configured for bearing against said remaining portions thereby limiting movement of the shaft in said introduction direction.

2. The middle ear prosthesis according to claim 1 wherein said stop member is a laterally projecting member.

3. The middle ear prosthesis according to claim 1 wherein said stop member is ring disk.

4. The middle ear prosthesis according to claim 1 wherein said stop member is mounted on the shaft thereby defining said distal end on a first side thereof and an upper portion on a second side thereof, said upper portion being configured to protrude inside a cavity of the middle ear.

5. The middle ear prosthesis according to claim 1 wherein said stop member is further configured to indicate to a surgeon conducting a medical procedure a location of said shaft with respect to said aperture.

6. The middle ear prosthesis according to claim 1 wherein said stop member is mounted on said coupling portion.

7. The middle ear prosthesis according to claim 1 any one of wherein said stop member is mount coupling portion and said shaft.

8. The middle ear prosthesis according to claim 1 wherein said stop member is adjustably mounted on said shaft.

9. The middle ear prosthesis according to claim 8 wherein said stop member is provided with fastening means, for securing said stop member in a desired location with respect to a length of said shaft.

10. An addition for a middle ear prosthesis for mounting in a middle ear having ossicles including remaining portions of a stapes bone with a footplate, the middle ear being disposed adjacent to an inner ear having an oval window with a membrane, the middle ear prosthesis including a shaft configured for the introduction into the inner ear through an aperture formed in the footplate of the remaining portions and in the membrane, by moving the shaft in an introduction direction, said shaft being configured for transmitting sound wave vibrations to the inner ear; and a coupling portion for coupling said shaft to at least one of the ossicles so as to allow transmitting thereby sound wave vibrations from the at least one of the ossicles to said shaft, the addition comprising:
a stop member mountable on the middle ear prosthesis thereby defining a distal end of the shaft configured for introduction into the inner ear, said stop member being configured for bearing against the remaining portions thereby limiting movement of the shaft in the introduction direction.

11. The addition according to claim 10 wherein said stop member is a laterally projecting member.

12. The addition according to claim 10 wherein said stop member is ring disk.

13. The addition according to claim 10 wherein said stop member is a rubber stretch ring.

14. The addition according to claim 10 wherein said stop member is configured to be mounted on the shaft thereby defining the distal end on a first side thereof and an upper portion on a second side thereof, the upper portion being configured to protrude inside the cavity of the middle ear.

15. The addition according to claim 10 wherein said stop member is further configured to indicate to a surgeon conducting a medical procedure a location of the shaft with respect to the aperture.

16. The addition according to claim 10 wherein said stop member is configured to be mounted on the coupling portion.

17. The addition according to claim 10 wherein said stop member is configured to be mounted between said coupling portion and said shaft.

18. The addition according to claim 10 wherein said stop member is configured to be adjustably mounted on the shaft.

19. The addition according to claim 18 wherein said stop member is provided with fastening means, for securing said stop member in a desired location with respect to a length of said shaft.
